(19) United States
(12) Patent Application Publication
(10) Pub. No.: US 2024/0424000 A1
(43) Pub. Date: Dec. 26, 2024

(54) LONG-ACTING INTRA-ARTICULAR DOSAGE FORMS CONTAINING FLUTICASONE PROPIONATE AND USE THEREOF

(71) Applicant: Eupraxia Pharmaceuticals Inc., Victoria (CA)

(72) Inventors: James Helliwell, Victoria (CA); Amanda Malone, Victoria (CA); James Price, Qualicum Beach (CA); Troy Loss, Vancouver (CA); Michael Gaines, Coquitlam (CA)

(21) Appl. No.: 18/753,995

(22) Filed: Jun. 25, 2024

Related U.S. Application Data
(60) Provisional application No. 63/510,309, filed on Jun. 26, 2023.

(51) Int. Cl. A61K 31/56; A61K 9/00; A61K 9/50; A61K 47/26; A61K 47/38; A61P 19/02; A61P 29/00

(52) U.S. Cl. CPC A61K 31/56; A61K 9/0019; A61K 9/5031; A61K 47/26; A61K 47/38; A61P 19/02; A61P 29/00

(57) ABSTRACT

Disclosed herein is a dosage form of fluticasone propionate for intra-articular administration to a patient with osteoarthritis in a joint.

### CROSS-REFERENCE TO RELATED APPLICATIONS

This application claims the benefit of priority to U.S. Provisional Application No. 63/510,309 filed Jun. 26, 2023, which application is hereby incorporated by reference in its entirety.

### BACKGROUND

### Technical Field

This disclosure relates to long-acting intra-articular injections of a sustained release form of fluticasone propionate and its therapeutic use, including for managing symptoms of osteoarthritis.

### Description of the Related Art

Osteoarthritis (OA) of the knee is a leading cause of lower extremity disability around the world. Treatment guidelines are aimed at symptoms management. Intra-articular (IA) corticosteroid injections such as triamcinolone acetonide (TCA) are conditionally recommended for symptom management (Kolasinski et al., Arthritis & Rheumatology, pp. 220-223, 72 (2), 2019). However, currently available corticosteroids are suboptimal due to their limited duration of efficacy and risk of systemic side effects (Juni et al., Cochrane Database Syst. Rev., 10, 2015). Longer IA residence time is expected to provide increased clinical benefit by extending the duration of efficacy and reducing the frequency of injections; however, only one extended-release corticosteroid (Zilretta®) is approved to date.

The local safety of IA corticosteroids continues to be debated. Data published in 2017 suggested that chronic exposure of TCA administered every 12 weeks for 2 years led to increased cartilage loss (McAlindon et al, J. Am. Med. Assoc., pp. 1967-1975, 317 (19), 2017). A more recent study evaluating the safety of IA cortisone or hyaluronic acid (HA) over 7-years concluded that IA corticosteroids were not associated with an increased risk of knee OA progression compared to HA (Bucci et al., Arthritis Rheum., 72 (Suppl 10), 2020).

There is a need for corticosteroid therapy that provides greater duration of localized efficacy with fewer systemic side effects such as glucose alterations and cortisol suppression.

### BRIEF SUMMARY

Fluticasone propionate microparticles of the present disclosure are capable of localized controlled release to optimize the pharmacokinetics (PK) of FP. Controlled release is achieved by coating FP crystals of well-defined geometries and dimensions with a thin membrane of cured polyvinyl alcohol (PVA). The combination of FP crystal and the thin PVA membrane provides fine control of FP's release rate via diffusion. The structure and the release mechanism of the PVA-coated FP crystals are described in more detail in U.S. Pat. No. 9,987,233.

Non-clinical studies evaluating PK and local safety of IA injection of long-acting coated fluticasone propionate microparticles (also referred to herein as “EP-104IAR”), including cartilage health, have been previously disclosed (Malone et al. Osteoarthritis and Cartilage Open, 3(4), 2021). These data indicated that the prolonged local residence time of EP-104IAR had no impact on cartilage health. Safety and PK data generated in a Phase I trial in 32 patients (24 were on active) with OA of the knee were consistent with nonclinical findings and supported continued development of EP-104IAR.

Disclosed herein are topline results from a Phase II trial evaluating the efficacy of EP-104IAR in 318 patients with OA of the knee. The primary and secondary endpoints were achieved, as described herein in more detail. The results support the aim of EP-104IAR, which is to maximize IA residence time while limiting systemic exposure, providing a greater duration of efficacy with fewer systemic side effects such as glucose alterations and cortisol suppression.

One embodiment thus provides a dosage form comprising fluticasone propionate, wherein the dosage form provides, after a single intra-articular injection to a subject, a maximum blood plasma concentration (Cₘₐₓ) of fluticasone propionate in the range of about 5-600 pg/mL, or 30-200 pg/mL, in the subject and a tₘₐₓ within the range of about 2 hours to 2 days, and wherein the fluticasone propionate is in the form of a plurality of microparticles, each microparticle comprising a crystal core of fluticasone propionate coated with a polyvinyl alcohol membrane.

Unless otherwise specified, the term “Cₘₐₓ” refers to the maximum plasma concentration of a drug achieved after administration to a subject. The term “tₘₐₓ” refers to the time at which the Cₘₐₓ is observed.

As used herein, reference to “about” a value herein includes (and describes) embodiments that are directed to that value per se. In certain embodiments, the term “about” includes the indicated amount±20%. In other embodiments, the term “about” includes the indicated amount±10%. In certain other embodiments, the term “about” includes the indicated amount and a range of −20% through +25% of the indicated amount.

As used herein, a “patient,” or “subject,” to be treated by methods according to various embodiments may mean either a human or a non-human animal, such as primates, mammals, and vertebrates.

In more specific embodiments, the dosage form comprises about 11 mg to 30 mg of fluticasone propionate. In even more specific embodiment, the dosage form comprises about 25 mg of fluticasone propionate.

In other more specific embodiments, the dosage form provides a plasma concentration of fluticasone propionate in the range of about 1 to 150 pg/mL, or about 30 to 120 pg/mL for at least 24 weeks.

In other more specific embodiments, the dosage form provides a half-life of fluticasone propionate of at least 12, 16, 18, 20, 22, 24, 28, 32, 36 or 40 weeks.

In other more specific embodiments, the dosage form provides a half-life of fluticasone propionate of at least 26 weeks.

In yet other more specific embodiments, the dosage form provides a mean serum concentration of cortisol in the subject of about 250 nmol/L or more for a period of at least 24 weeks. In other more specific embodiments, the dosage form provides a mean serum concentration of cortisol in the subject of about 250 nmol/L or more for a period of at least 12 weeks, or at least 16 weeks, or at least 18 weeks, or at least 20 weeks, or at least 22 weeks, or at least 24 weeks, or at least 28 weeks, or at least 32 weeks, or at least 36 weeks, or at least 40 weeks.

In various embodiments, the subject has moderate OA pain with WOMAC pain scores ranging from 3.5 to 6.5, and the dosage form causes a decrease in the WOMAC pain score in the subject. In other embodiments, the subject has OA with WOMAC pain scores ranging from 3.5 to 9.5, and the dosage form causes a decrease in the WOMAC pain score in the subject.

In various embodiments, the plurality of the microparticles in the dosage form have a size distribution of (i) 90% of the total mass (D₉₀) are no larger than 250 microns; (ii) 50% of the total mass (D₅₀) have a mean size in the range of 120-160 microns; (iii) 10% of the total mass (D₁₀) are less than 65 microns.

In various embodiments, the plurality of the microparticles in the dosage form have a size distribution such that: (i) D₁₀ of the microparticles in the dosage form is at least 65 microns; (ii) D₅₀ of the microparticles in the dosage form ranges from 120 microns to 160 microns; and (iii) D₉₀ of the microparticles in the dosage form is less than or equal to 250 microns, with the provisos that D₁₀ is less than D₅₀, and D₉₀ is greater than D₅₀.

### BRIEF DESCRIPTION OF THE SEVERAL VIEWS OF THE DRAWINGS

FIG. 1 shows plots comparing changes from baseline of WOMAC Pain for EP-104IAR versus vehicle over a period of 24 weeks;

FIG. 2 shows plots comparing changes from baseline of WOMAC Function for EP-104IAR versus vehicle over a period of 24 weeks;

FIG. 3 shows plots comparing the AUC for WOMAC Pain for EP-104IAR versus vehicle over a period of 24 weeks;

FIG. 4 shows plots comparing the percentage of OMERACT-OARSI strict responders for EP-104IAR versus vehicle over a period of 24 weeks;

FIG. 5 shows plots comparing changes from baseline of WOMAC Pain Moderate WOMAC Pain for EP-104IAR versus vehicle over a period of 24 weeks;

FIG. 6 shows plots comparing the percentage of OMERACT-OARSI Strict Responders Moderate WOMAC Pain at baseline for EP-104IAR versus vehicle over a period of 24 weeks;

FIGS. 7a and 7b show plots comparing the percentage of patients with WOMAC Pain ≤1 and ≤2 Moderate WOMAC Pain at baseline for EP-104IAR versus vehicle over a period of 24 weeks;

FIG. 8 shows a geometric mean plasma concentration of curve for FP over the 24-week period;

FIG. 9 shows a plot of mean serum concentration of cortisol over a period of 24 weeks;

FIG. 10a shows a plot of mean serum glucose level for diabetic population over a period of 24 weeks;

FIG. 10b shows a plot of mean serum glucose level for entire patient population over a period of 24 weeks; and

FIG. 11 shows plots comparing the plasma FP concentrations for the Phase I versus Phase II products over a period of 24 weeks following intra-articular injection to OA subjects.

### DETAILED DESCRIPTION

As described in further detail herein, the present disclosure provides a dosage form of long-acting fluticasone propionate (FP) for IA injection. Based on the clinical trial results, the dosage form provides extended and steady release of FP, as evidenced by the plasma FP levels, which remained below a level that may result in any clinically significant cortisol suppression. Patients with moderate OA pain experienced sustained pain relief.

### Clinical Trial Protocol

In this Phase II, randomized, double-blind, vehicle-controlled parallel-group study (NCT04120402), eligible subjects with qualifying knee OA pain were randomized about 1:1 to receive a single IA dose of EP-104IAR 25 mg, or vehicle and followed up for 24 weeks. The study enrolled male and females, ≥40 years, with a diagnosis of primary knee OA (per ACR clinical and radiological criteria), with a Kellgren-Lawrence Grade of 2-3 and OA symptoms for ≥6 months.

Potential participants completed a 2-week washout/baseline period, from which their baseline and qualifying pain was determined. Qualifying knee pain was defined as weekly Western Ontario and McMaster Universities Osteoarthritis Index (WOMAC®) Pain subscale scores ≥4.0 to ≤9.0 (out of 10) which did not vary by >3 points. Subjects with bilateral knee OA were required to have WOMAC Pain scores of ≤6.0 (out of 10) in their non-Index knee.

Subjects were administered a single dose of either EP-104IAR or vehicle and recorded weekly WOMAC pain and monthly WOMAC total Index measurements using the provided ePRO device. Safety was assessed via adverse events, vital signs, clinical laboratory evaluations (including serum cortisol and ACTH stimulation testing), and physical/knee examinations. Blood samples for measurement of FP were collected at every visit.

Rescue medication (acetaminophen up to 3 g/day) was permitted. Subjects were instructed to record its use on their devices and refrain from use for 12 hours prior to completing the WOMAC questionnaire.

The primary efficacy endpoint was the difference in change from baseline between EP-104IAR and vehicle in WOMAC Pain at Week 12. Analysis was performed using a mixed-effects model for repeated measures (MMRM) in the intention to treat (ITT) population.

Key secondary endpoints were analyzed using analogous methods using a step-down hierarchical testing procedure to avoid multiplicity issues. Secondary endpoints included: (1) difference between treatments in WOMAC Function subscale at Week 12, (2) difference between treatments in the area under the WOMAC Pain-time curve to 12 weeks, (3) WOMAC Pain at Week 24, and (4) the difference between treatments in OMERACT-OARSI strict responders (Pham, et al., 2004) at Week 12.

The study comprised 318 treated subjects (n=163 EP-104IAR, n=155 vehicle), median age=64 yrs, 58% female, 99% Caucasian. 304 subjects completed the study. 14 subjects withdrew prior to the 12-week visit (the majority for ‘Withdrawal by Subject’).

### Clinical Trial Results

In this study, a single dose of EP-104IAR 25 mg provided statistically significant pain relief at 12 weeks compared to vehicle-control, thereby meeting the primary endpoint. Additionally, three key secondary endpoints were statistically significantly different for WOMAC function at Week 12, area under the WOMAC Pain-time curve at Week 12, and composite pain and function OMERACT-OARSI response at Week 12. Furthermore, durability of response was observed with statistically significant differences in WOMAC Pain subscale scores between EP-104IAR and vehicle out to Week 14.

EP-104IAR 25 mg was safe, generally well-tolerated and resulted in low but sustained plasma levels for the entire 24-week study period. The safety and efficacy of EP-104IAR will be further evaluated in Phase III trials.

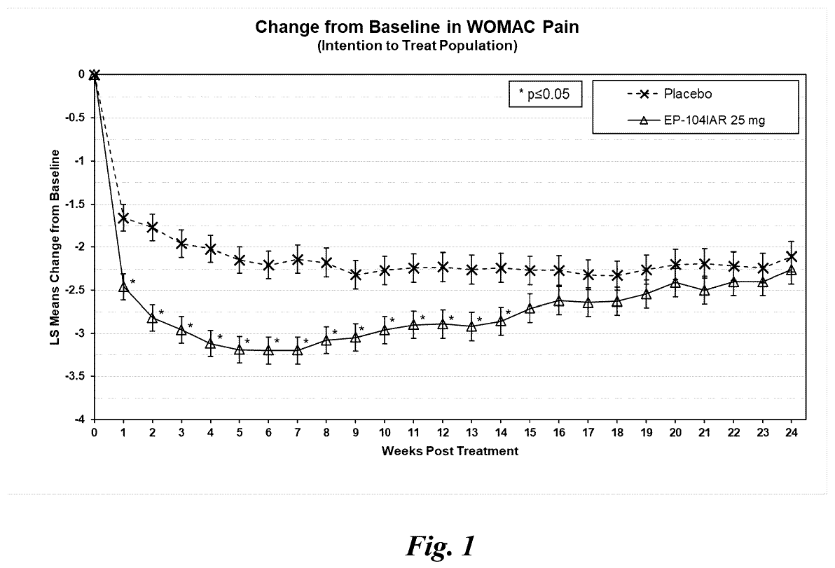

The primary endpoint was met, as shown in FIG. 1. In particular, EP-104IAR demonstrated a statistically significant difference compared to vehicle in WOMAC Pain at Week 12 (least-squares mean change from baseline: −2.89 versus −2.23; p=0.004). FIG. 1 further demonstrates significant, durable and meaningful pain relief up to 14 weeks.

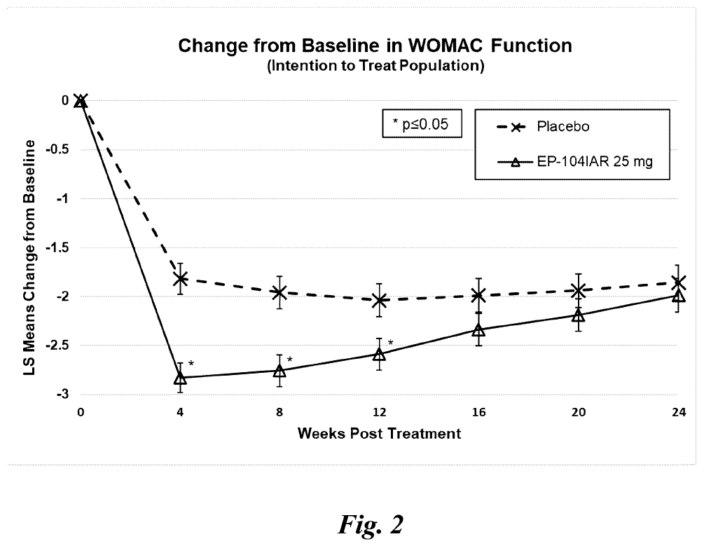

Three secondary endpoints were also met. First, EP-104IAR provided statistically significant improvement in the difference in change from baseline between EP-104IAR and vehicle in WOMAC Function at Week 12 (least-squares mean change from baseline: −2.59 versus −2.04; p=0.014). See FIG. 2.

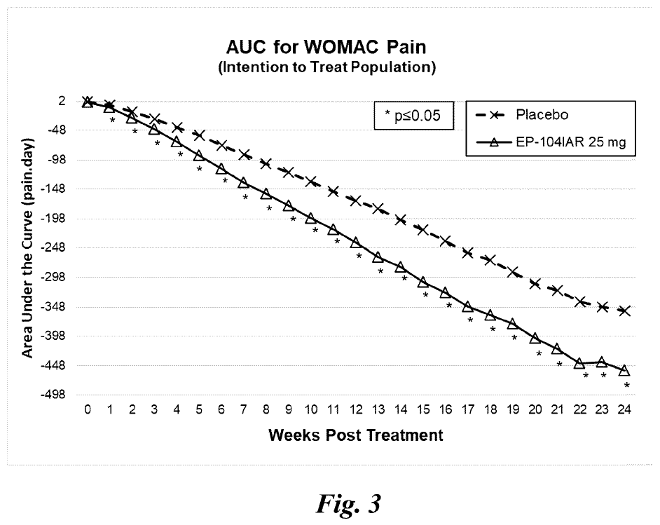

Furthermore, as shown in FIG. 3, EP-104IAR provided statistically significant reduction in the area under the WOMAC Pain-time curve to Week 12 (Week 12 EP-104IAR: −239 versus −167; p<0.001). FIG. 3 further shows that EP-104IAR provides better average pain relief than placebo to 24 weeks. EP-104IAR thus provided statistically significant improvement in the difference in change from baseline between EP-104IAR and vehicle in WOMAC Pain at Week 24 (least-squares mean change from baseline: −462 versus −352; p=0.012).

Eighty-seven (87) (56%) of the EP-104IAR subjects met the OMERACT-OARSI strict responder definition (assessed through either pain or function) at 12 weeks post-dose compared to 61 (43%) of vehicle subjects. (p=0.028).

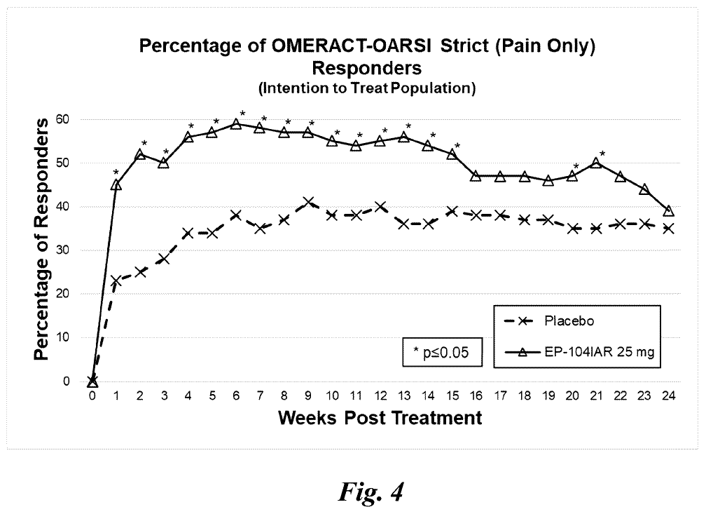

An alternative version of OMERACT-OARSI strict responder relies on pain alone. Under this definition, a strict responder is a patient that experienced a clinically meaningful impact in their pain response. Equally important to treating pain, it is also desirable to achieve a level of pain relief for patients that allows them to be most comfortable. A strict pain responder is defined as a 50% or greater improvement in their WOMAC pain score from baseline, with an absolute change of at least two points on the WOMAC scale. Patients need to achieve both of those to be deemed a strict responder. As shown in FIG. 4, strict responders experienced significant improvements in pain.

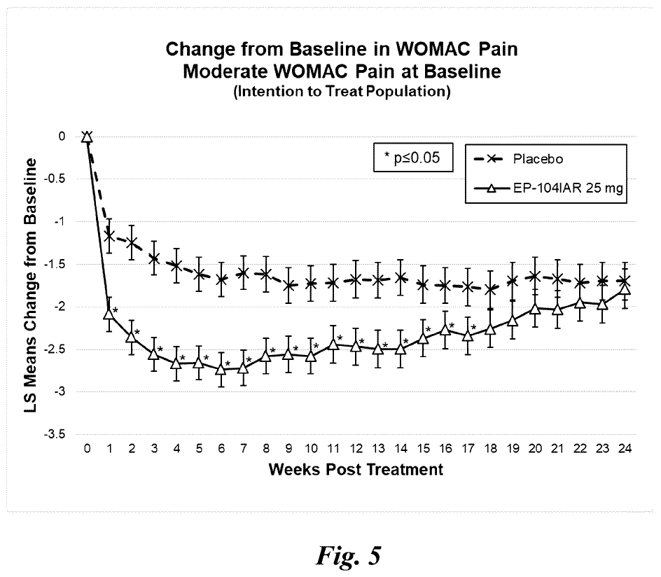

The majority of patients (68% of the study population, n=214) had moderate OA pain, defined as those patients with WOMAC pain scores ranging from 3.5 to 6.5. For these moderate OA pain patients, EP-104IAR provided statistically significant improvements in the difference in change from baseline between EP-104IAR and vehicle in WOMAC Pain at all weeks up to Week 17 (least-squares mean change from baseline: −2.34 versus −1.77; p=0.026). See FIG. 5.

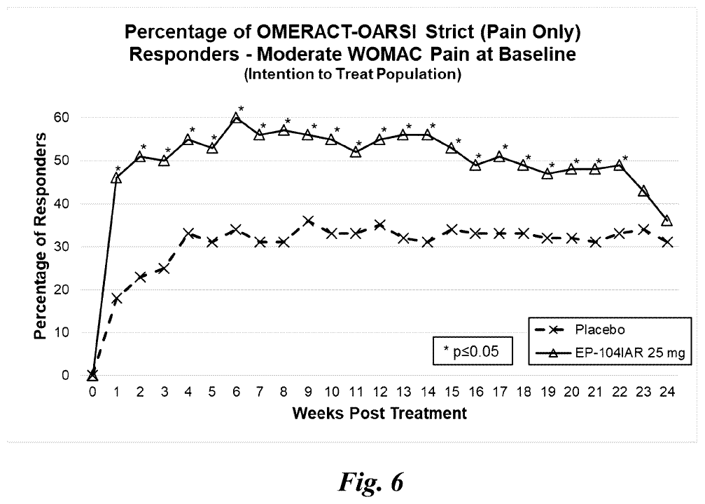

FIG. 6 shows that OMERACT-OARSI Strict Responders (pain alone) in moderate OA pain patients (about ⅔ of all the patients) experienced 22 weeks of clinically meaningful and significant improvements in pain.

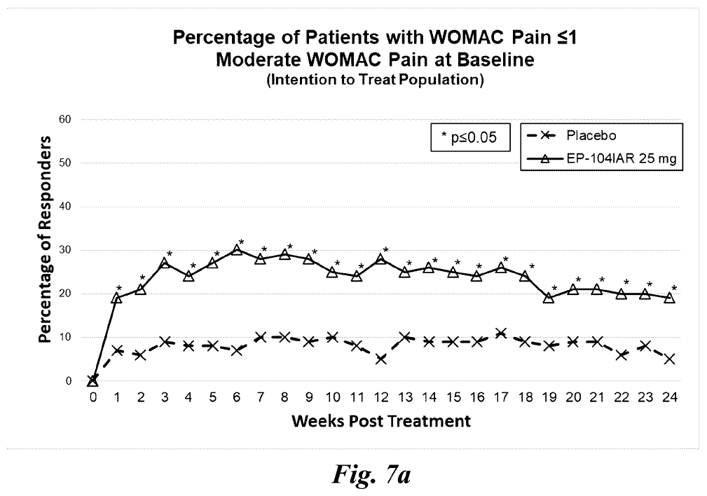

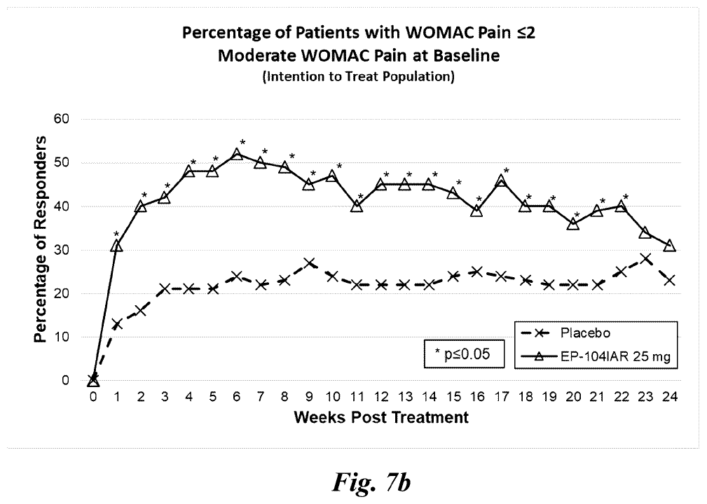

FIGS. 7a and 7b show that significant percentages (about 40%) of moderate OA pain patients maintained WOMAC pain stores of less than 1 or less than 2, respectively, for 22 to 24 weeks. This indicates that near complete pain relief could be achieved for a sustained period of time.

The majority of treatment-emergent adverse events (AEs) were mild-moderate in severity, as summarized in Table 1. The most common AEs (occurring in >5% of subjects in either treatment arm) were arthralgia, COVID-19, nasopharyngitis, influenza and influenza-like illness. In the Safety Population, two (2) AEs in two (2) subjects led to discontinuation: Spinal Column Injury and Arthralgia (worsening of pain left (non-index) knee).

TABLE 1
Adverse Events by Treatment Group (Safety Population)

| | EP-104IAR | | | | | |
|---|---|---|---|---|---|---|
| | 25 mg | | Placebo | | Overall | |
| | N = 163 | | N = 155 | | N = 318 | |
| Subjects with at least 1 TEAE | 106 | (65.0%) | 89 | (57.4%) | 195 | (61.3%) |
| Mild | 47 | (28.8%) | 33 | (21.3%) | | |
| Moderate | 57 | (35.0%) | 55 | (35.5%) | | |
| Severe | 2 | (1.2%) | 1 | (0.6%) | | |
| Subjects with at least 1 Serious | 4 | (2.5%) | 1 | (0.6%) | 5 | (1.6%) |
| TEAE | | | | | | |
| Subjects with study medication- | 15 | (9.2%) | 11 | (7.1%) | 26 | (8.2%) |
| related TEAE | | | | | | |
| Subjects with at least 1 TEAE | 2 | (1.2%) | 0 | | 2 | (0.6%) |
| leading to withdrawal | | | | | | |

### Pharmacokinetic Results

The plasma concentrations of fluticasone propionate were measured at various intervals for 24 weeks, and the measured plasma concentrations (pg/mL) are summarized in Table 2 below.

TABLE 2

| | | % | | | | | | |
|---|---|---|---|---|---|---|---|---|
| | | release* | Gmean | Min | Q1 | Median | Q3 | Max |
| 2 | h | 0.2 | 55 | 1 | 18 | 86 | 149 | 602 |
| 2 | d | 1.1 | 72 | 6 | 37 | 76 | 147 | 508 |
| 2 | w | 4.0 | 38 | 1 | 26 | 46 | 67 | 193 |
| 4 | w | 7.0 | 34 | 1 | 25 | 42 | 54 | 137 |
| 8 | w | 12.8 | 31 | 1 | 24 | 37 | 51 | 159 |
| 12 | w | 18.6 | 32 | 1 | 22 | 39 | 54 | 167 |
| 18 | w | 27.1 | 30 | 1 | 21 | 35 | 49 | 128 |
| 24 | w | 33.5 | 25 | 1 | 19 | 28 | 43 | 120 |

*Proportion of drug released based on preliminary data.

Dose delivered: Mean of 26.3 mg, SD of 2.9 mg, median of 27.2 mg, IQR of 25.2 to 28.2 mg, range of 11.5 to 30.0 mg.

Cₘₐₓ: Gmean of 90.1 pg/mL, CV of 126.1%, IQR of 38 to 189 pg/mL, max of 602 pg/mL.

tₘₐₓ: Median of 22.25 hours s, IQR of 2 hrs to 2 days

Half-life: estimated to be 36.78 weeks.

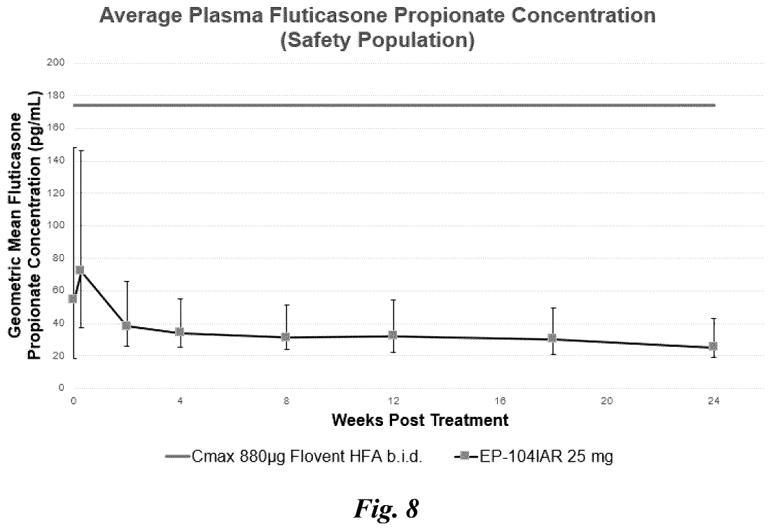

Thus, the EP-104IAR dosage form is capable of extended release to 24+ weeks with large systemic safety margin. FIG. 8 shows the geometric mean plasma concentration curve over the 24-week period, which is well below the average plasma concentration for Flovent, a daily-administered inhaled aerosol of fluticasone propionate. In particular, the average maximum plasma concentration (Cₘₐₓ) of FP was 89.3 pg/mL, at 2 days (tₘₐₓ) post dose. Following this initial peak, plasma concentrations decreased slowly and steadily over time and remained at low but detectable levels until the end of the study.

### Safety Profile

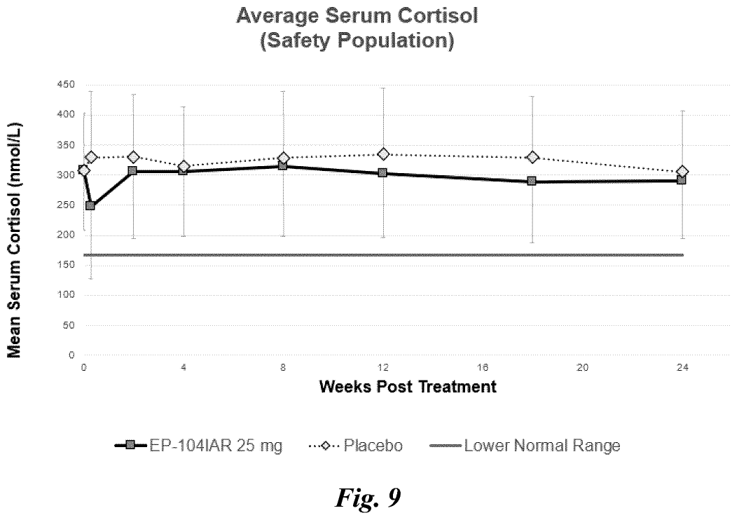

Serum cortisol is a key safety indicator and was monitored throughout the study. As shown in FIG. 9, average cortisol levels dropped to about 250 nmol/L in EP-104 subjects at day 2, but returned to about normal (pre-FP administration level) by 2 weeks post-dose. It is significant that the serum cortisol levels (other than day 2) were comparable with placebo throughout the monitoring, despite the fact that fluticasone propionate was present in the plasma for 24 weeks.

ACTH stimulation testing demonstrated that no subjects experienced a failed ACTH test accompanied with the signs and/or symptoms of adrenal insufficiency following EP-104IAR administration. There were no clinically significant differences in any laboratory assessments between the treatment groups.

Thus, dosage forms of the present disclosure can provide an average serum cortisol of 250 nmol/L or more, which is above the lower normal range. In some embodiments, this safety profile enables repeat or bilateral dosing, i.e., treatment of both knees at once for the 70% of OA patients that suffer from bilateral disease—instead of treating only one knee and leaving a patient in discomfort with pain in the untreated knee.

Serum glucose is also an important marker in the OA disease state. It is understood that steroids can suppress not only cortisol levels but may also affect glycogen levels in the liver by increasing the release of glucose.

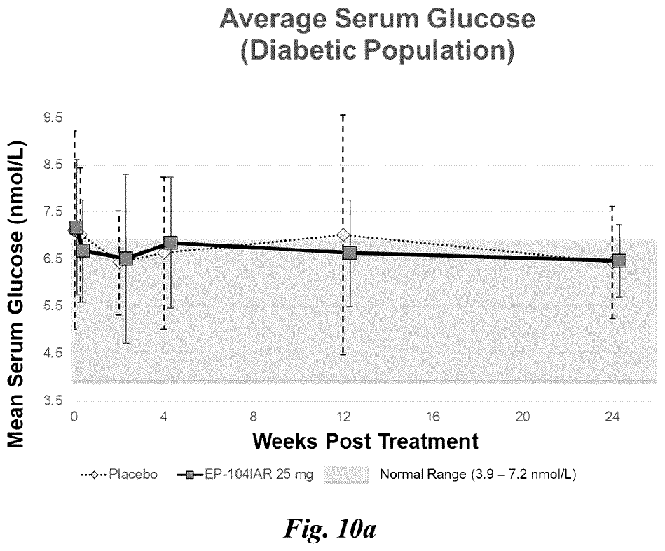

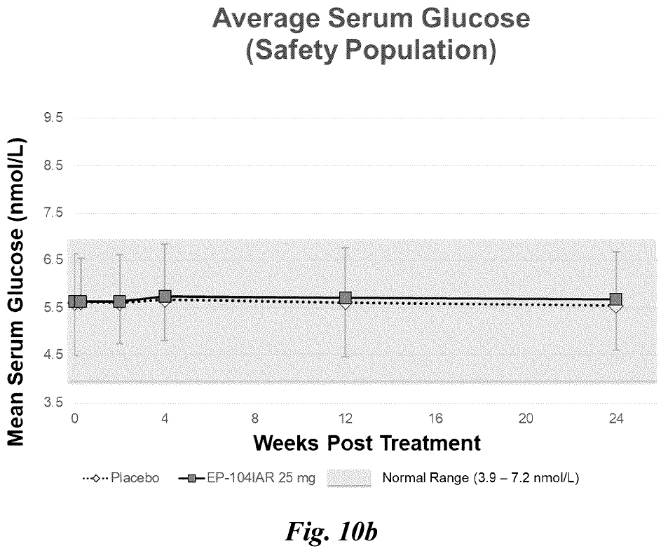

Such glucose derangement can make it unsafe for diabetic patients. FIG. 10a shows the mean serum glucose level for the diabetic patients in the trial, of which there were 13 in each of the active and placebo arms. While baseline serum glucose levels were higher for the diabetic patients, as would be expected in any diabetic population, median change from baseline in glucose on Day 3 for subjects who received EP-104IAR was similar to that in subjects without a history of NIDDM (−0.10 mmol/L and 0.00 mmol/L respectively). This study indicates that, for the 30% of the OA patients that are also diabetic, dosage forms of the present disclosure can provide a durable, efficacious and systemically safe therapeutic that is compatible with glucose-controlling diabetic treatments.

FIG. 10b shows the mean serum glucose for the trial population as whole. As shown, the dosage form had no impact on serum glucose levels.

### EP-104IAR Preparation

The manufacturing process for the Phase II product comprises two major steps: first, production of bulk drug substance and, then, production of EP-104IAR Powder. The commercially sourced active pharmaceutical ingredient (API), FP, was recrystallized, wet milled and sieved to achieve consistent and larger crystal sizes suitable for the subsequent application of the polyvinyl alcohol (PVA) polymer coating. The large crystals, i.e., bulk drug substance, were then coated, cured, irradiated and aseptically filled into vials to form the EP-104IAR Powder (“Phase II products”).

The resulting dosage form includes a plurality of microparticles containing fluticasone propionate, wherein each microparticle comprises a crystal core of fluticasone propionate coated with a polyvinyl alcohol membrane. As explained below, the particle size distribution of the microparticles affects the release characteristics of the dosage form.

U.S. Patent Publication No. 2022/0168665 discloses a previous iteration of producing bulk drug substance and coated particles, which were utilized in a Phase I trial (“Phase I product”). Table 3 compares the particle size distributions of the Phase I and II products, in which the Phase II product is an embodiment of the present disclosure.

TABLE 3
Comparison of Particle Size for the Phase I and Phase II Products

| Test | Phase I Product | | Phase II Product |
|---|---|---|---|
| Particle size | | | |
| D₁₀ | 26.4 | μm | NLT 65 μm |
| D₅₀ | 64.5 | μm | 120-160 μm |
| D₉₀ | 100.2 | μm | NMT 250 μm |

Notes:
D₁₀ = 10% of particles less than value; D₅₀ = Mean size; D₉₀ = 90% of particles less than value; NLT = Not less than; NMT = Not more than.

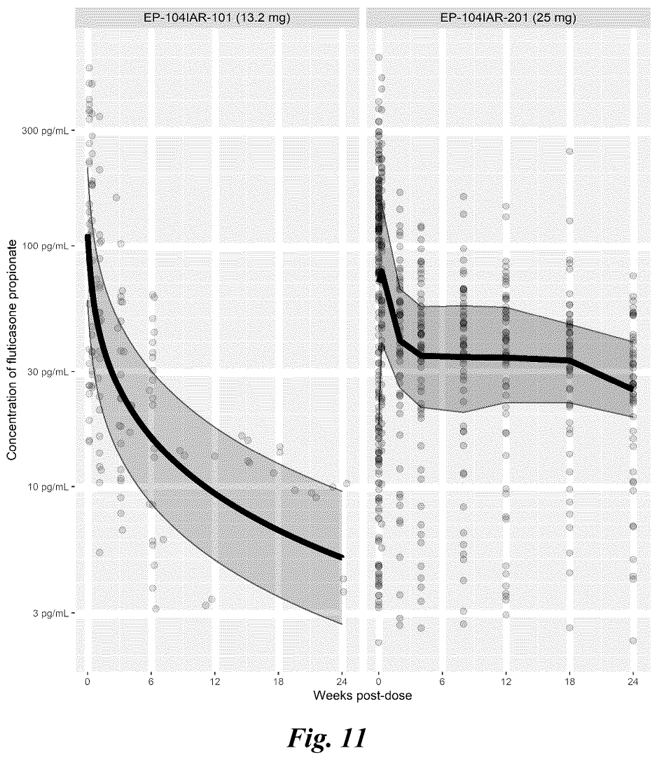

The differences in the size distributions between the Phase I and II products are believed to have caused the profound differences in the in vivo release, as evidenced in FIG. 11. As shown, the Phase II product, according to one embodiment described herein, demonstrated a steady (flat) and sustained plasma FP concentration over at least 24 weeks, a majority of the entire period of the clinical trial. In contrast, the Phase I product exhibited a decline in plasma FA concentration without the steady (flat) period exhibited by the Phase II product.

As used herein, the term D₁₀ represents the particle size below which 10 mass % of microparticles are sized within a particle size distribution of microparticles. In some embodiments the D₁₀ of microparticles in the dosage form is at least 65 μm (i.e., not less than 65 μm). In some embodiments the D₁₀ of microparticles in the dosage form ranges from 65 μm to 90 μm, or from 65 μm to 70 μm, or from 70 μm to 75 μm, or from 75 μm to 80 μm, or from 80 μm to 85 μm, or from 85 μm to 90 μm.

In some embodiments the particle size distribution of the microparticles in the dosage form is such that 10 mass % of the microparticles have a particle size of less than about 65 μm (i.e., D₁₀˜65 microns). In some embodiments the particle size distribution of the microparticles in the dosage form is such that 10 mass % of the microparticles have a particle size of less than about 70 μm (i.e., D₁₀˜70 μm). In some embodiments the particle size distribution of the microparticles in the dosage form is such that 10 mass % of the microparticles have a particle size of less than about 75 μm (i.e., D₁₀˜75 μm). In some embodiments the particle size distribution of the microparticles in the dosage form is such that 10 mass % of the microparticles have a particle size of less than about 80 μm (i.e., D₁₀˜80 μm). In some embodiments the particle size distribution of the microparticles in the dosage form is such that 10 mass % of the microparticles have a particle size of less than about 85 μm (i.e., D₁₀˜85 μm). In some embodiments the particle size distribution of the microparticles in the dosage form is such that 10 mass % of the microparticles have a particle size of less than about 90 μm (i.e., D₁₀˜90 μm).

As used herein, the term D₅₀ represents the median particle size of the microparticles in the dosage form—i.e., the particle size below which 50 mass % of the microparticles are sized and above which 50 mass % of the microparticles are sized. In some embodiments the D₅₀ of microparticles in the dosage form is at least 120 μm. In some embodiments the D₅₀ of microparticles in the dosage form is at least 120 μm. In some embodiments the D₅₀ of microparticles in the dosage form is no more than 160 μm. In some embodiments the D₅₀ of microparticles in the dosage form ranges from 120 μm to 160 μm, or from 120 μm to 125 μm, or from 125 μm to 130 μm, or from 135 μm to 140 μm, or from 140 μm to 145 μm, or from 145 μm to 150 μm, or from 150 μm to 155 μm, or from 155 μm to 160 μm. In some embodiments the D₅₀ of microparticles in the dosage form ranges from 125 μm to 135 μm.

In some embodiments the particle size distribution of the microparticles in the dosage form is such that the median particle size (D₅₀) is about 120 μm. In some embodiments the particle size distribution of the microparticles in the dosage form is such that the median particle size (D₅₀) is about 125 μm. In some embodiments the particle size distribution of the microparticles in the dosage form is such that the median particle size (D₅₀) is about 130 μm. In some embodiments the particle size distribution of the microparticles in the dosage form is such that the median particle size (D₅₀) is about 135 μm. In some embodiments the particle size distribution of the microparticles in the dosage form is such that the median particle size (D₅₀) is about 140 μm. In some embodiments the particle size distribution of the microparticles in the dosage form is such that the median particle size (D₅₀) is about 145 μm. In some embodiments the particle size distribution of the microparticles in the dosage form is such that the median particle size (D₅₀) is about 150 μm. In some embodiments the particle size distribution of the microparticles in the dosage form is such that the median particle size (D₅₀) is about 155 μm. In some embodiments the particle size distribution of the microparticles in the dosage form is such that the median particle size (D₅₀) is about 160 μm.

As used herein, the term D₉₀ represents the particle size below which 90 mass % of microparticles are sized within a particle size distribution of microparticles. In some embodiments the D₉₀ of microparticles in the dosage form is less than or equal to 250 μm (i.e., not more than 250 μm). In some embodiments the D₉₀ of microparticles in the dosage form ranges from 180 μm to 250 μm, or from 180 μm to 185 μm, or from 185 μm to 190 μm, or from 190 μm to 195 μm, or from 195 μm to 200 μm, or from 200 μm to 205 μm, or from 205 μm to 210 μm, or from 210 μm to 215 μm, or from 215 μm to 220 μm, or from 225 μm to 230 μm, or from 230 μm to 235 μm, or from 235 μm to 240 μm, or from 240 μm to 245 μm, or from 245 μm to 250 μm.

In some embodiments the particle size distribution of the microparticles in the dosage form is such that 90 mass % of the microparticles have a particle size of less than about 180 μm (i.e., D₉₀˜180 microns). In some embodiments the particle size distribution of the microparticles in the dosage form is such that 90 mass % of the microparticles have a particle size of less than about 185 μm (i.e., D₉₀˜185 microns). In some embodiments the particle size distribution of the microparticles in the dosage form is such that 90 mass % of the microparticles have a particle size of less than about 190 μm (i.e., D₉₀˜190 microns). In some embodiments the particle size distribution of the microparticles in the dosage form is such that 90 mass % of the microparticles have a particle size of less than about 195 μm (i.e., D₉₀˜195 microns). In some embodiments the particle size distribution of the microparticles in the dosage form is such that 90 mass % of the microparticles have a particle size of less than about 200 μm (i.e., D₉₀˜200 microns). In some embodiments the particle size distribution of the microparticles in the dosage form is such that 90 mass % of the microparticles have a particle size of less than about 205 μm (i.e., D₉₀˜205 microns). In some embodiments the particle size distribution of the microparticles in the dosage form is such that 90 mass % of the microparticles have a particle size of less than about 210 μm (i.e., D₉₀˜210 microns). In some embodiments the particle size distribution of the microparticles in the dosage form is such that 90 mass % of the microparticles have a particle size of less than about 220 μm (i.e., D₉₀˜220 microns). In some embodiments the particle size distribution of the microparticles in the dosage form is such that 90 mass % of the microparticles have a particle size of less than about 225 μm (i.e., D₉₀˜225 microns). In some embodiments the particle size distribution of the microparticles in the dosage form is such that 90 mass % of the microparticles have a particle size of less than about 230 μm (i.e., D₉₀˜230 microns). In some embodiments the particle size distribution of the microparticles in the dosage form is such that 90 mass % of the microparticles have a particle size of less than about 235 μm (i.e., D₉₀˜235 microns). In some embodiments the particle size distribution of the microparticles in the dosage form is such that 90 mass % of the microparticles have a particle size of less than about 240 μm (i.e., D₉₀˜240 microns). In some embodiments the particle size distribution of the microparticles in the dosage form is such that 90 mass % of the microparticles have a particle size of less than about 245 μm (i.e., D₉₀˜245 microns). In some embodiments the particle size distribution of the microparticles in the dosage form is such that 90 mass % of the microparticles have a particle size of less than about 250 μm (i.e., D₉₀˜250 microns).

Table 4 summarizes the composition of the vehicle suitable for the embodiments according to the present disclosure, including the EP-104IAR drug product used in the clinical trial.

TABLE 4
Composition of EP-104IAR Vehicle

| | | Amount | |
|---|---|---|---|
| Component | Function | (mg) | % wt/v |
| Carboxymethylcellulose Sodium (Medium | Viscosity Builder | 37.5 | 0.75 |
| Viscosity, MW = ~250,000 g/mol), USP¹ | | | |
| Polysorbate 80, NF² | Suspending | 0.75 | 0.015 |
| | Agent | | |
| Sodium Chloride, USP | Isotonicity | 41.0 | 0.820 |
| Sodium Phosphate Dibasic Heptahydrate, USP | Buffer | 10.90 | 0.218 |
| Sodium Phosphate Monobasic Monohydrate, USP | Buffer | 1.30 | 0.026 |
| Water for Irrigation, USP | Diluent | QS³ to | QS to |
| | | 5.00 mL | 100% |

| | | |
|---|---|---|
| Total | 5.00 mL⁴ | 100 |

¹USP = United States Pharmacopeia
²NF = National Formulary
³QS = Quantum sufficit
⁴Vials are filled with 6 mL to ensure 5 mL can be withdrawn and used to constitute the powder prior to injection.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety. Aspects of the embodiments can be modified, if necessary to employ concepts of the various patents, applications and publications to provide yet further embodiments.

These and other changes can be made to the embodiments in light of the above-detailed description. In general, in the following claims, the terms used should not be construed to limit the claims to the specific embodiments disclosed in the specification and the claims, but should be construed to include all possible embodiments along with the full scope of equivalents to which such claims are entitled. Accordingly, the claims are not limited by the disclosure.

1. A dosage form comprising fluticasone propionate, wherein the dosage form provides, after a single intra-articular injection to a subject, a maximum blood plasma concentration (Cₘₐₓ) of fluticasone propionate in the range of about 5-600 pg/mL in the subject and a tₘₐₓ within the range of about 2 hours to 2 days, and wherein the fluticasone propionate is in the form of a plurality of microparticles, each microparticle comprising a crystal core of fluticasone propionate coated with a polyvinyl alcohol membrane.

2. The dosage form of claim 1, comprising about 11 mg to 30 mg of fluticasone propionate.

3. The dosage form of claim 1, comprising about 25 mg of fluticasone propionate.

4. The dosage form of claim 1, wherein the dosage form provides a plasma concentration of fluticasone propionate in the range of about 1 to 150 pg/mL for at least 24 weeks.

5. The dosage form of claim 1, wherein the dosage form provides a half life of fluticasone propionate of at least 12 weeks.

6. The dosage form of claim 1, wherein the dosage form provides a mean serum concentration of cortisol in the subject of 250 nmol/L or more for a period of at least 24 weeks.

7. The dosage form of claim 1, wherein the plurality of the microparticles in the dosage form have a size distribution of (i) 90% of the total mass (D₉₀) are no larger than 250 microns; (ii) 50% of the total mass (D₅₀) have a mean size in the range of 120-160 microns; (iii) 10% of the total mass (D₁₀) are less than 65 microns.

8. The dosage form of claim 1, wherein the plurality of the microparticles in the dosage form have a size distribution such that: (i) D₁₀ of the microparticles in the dosage form is at least 65 microns; (ii) D₅₀ of the microparticles in the dosage form ranges from 120 microns to 160 microns; and (iii) D₉₀ of the microparticles in the dosage form is less than or equal to 250 microns, with the provisos that D₁₀ is less than D₅₀, and D₉₀ is greater than D₅₀.

9. The dosage form of claim 1, wherein the subject has moderate OA pain with WOMAC pain scores ranging from 3.5 to 6.5, and the dosage form causes a decrease in the WOMAC pain score in the subject.

10. The dosage form of claim 1, wherein the subject has OA with WOMAC pain scores ranging from 3.5 to 9.5, and the dosage form causes a decrease in the WOMAC pain score in the subject.

11. The dosage form of claim 1, which is capable of reducing or alleviating pain in a joint of the subject.

12. The dosage form of claim 1, which is capable of treating osteoarthritis in the subject.

13. The dosage form of claim 11, wherein the subject has a WOMAC pain scores ranging from 3.5 to 9.5 prior to the injection.

14. The dosage form of claim 13 wherein the subject has a WOMAC pain scores ranging from 3.5 to 6.5 prior to the injection.
